(19) United States
(12) Patent Application Publication
(10) Pub. No.: US 2025/0257512 A1
(43) Pub. Date: Aug. 14, 2025

(54) UVC-ASSISTED WASHING APPARATUS AND METHOD

(71) Applicant: CONTROLADORA MABE, S.A. DE C.V., Ciudad de Mexico (MX)

(72) Inventors: Santiago Alonso PLATA AMARILLAS, Queretaro (MX); Ramon Merino Cervantes, Queretaro (MX); Orlando Pedraza Otero, Queretaro (MX); Iván Perrusquía Hernández, Queretaro (MX); Juan Arturo Molina Cortéz, Queretaro (MX)

(21) Appl. No.: 18/808,546

(22) Filed: Aug. 19, 2024

(30) Foreign Application Priority Data
Feb. 8, 2024 (MX) A/2024/001789

(51) Int. Cl. D06F 35/00; A61L 2/10; A61L 2/24; A61L 2/26; C02F 1/32; C02F 103/00; D06F 39/08

(52) U.S. Cl. CPC D06F 35/008; A61L 2/10; A61L 2/24; A61L 2/26; C02F 1/325; D06F 39/083; A61L 2202/11; A61L 2202/122; A61L 2202/26; C02F 2103/002; C02F 2303/04

(57) ABSTRACT

The present invention relates to a washing method and household appliance for textiles in a household appliance, which offers the benefit of disinfection of clothing, the washing liquid, as well as the components of the washing machine that are in contact with the washing liquid and loading clothes through the use of UV-C radiation. Disinfection can be carried out during the washing and/or rinsing step through a predetermined sequence of turning on and off the radiation source. In addition to disinfection without the need to add temperature to the water at the inlet of the washing machine or inside it, there are the benefits of greater cleaning and less damage to clothing, shorter washing time, lower energy consumption, and avoiding the use of disinfectant additives for more ecological wash cycles.

### OBJECT OF THE INVENTION

The present invention relates to a system and method for disinfecting textiles, the washing liquid that is sent to the drain, as well as the components of the washing machine that are in contact with the washing liquid and the load of clothes through the use of ultraviolet light with wavelength C (UVC), depending on the location of the radiation source in the household appliance. In addition to disinfection, this method allows obtaining greater cleaning of the textiles, less washing time, less damage to the textiles, less energy consumption and more ecological washing cycles. The method works from a temperature of 15° C., so it avoids the use of high temperatures in the water that is fed to the inlet of the washing machine or inside it for disinfection, resulting in an efficient and economical process.

### BACKGROUND

In the state of the art, the use of methods for disinfecting textiles using UVC light is known, for example, document WO 2015/169348, which describes a front-loading clothes washing machine comprising a washing tub external to a swivel washing drum adapted to receive clothes; a recirculation circuit comprising a conduit for the washing liquid to flow, a seat to receive said UV source and a housing that includes a transparent wall to allow light to pass through. The UV lamp can be activated to remove organic matter during several cycles of the washing routine.

Document CN203270310 is also known, which discloses a top-loading washing machine that comprises an ultraviolet radiation generating device that in turn is coupled to a PLC. The method described in this invention comprises: putting the clothes in the tub, adding the amount of detergent and water to the set level, soaking the clothes, irradiating the clothes and the washing water with a UV generating device, rinsing and dehydrating the load. Optionally, the method comprises a final step of removing odors from clothing, using the UV generating device.

Another known document is CN101446020, which describes a top-loading washing machine comprising a housing; a water storage tank arranged in the housing to receive influent washing water to the outside; a washing tank arranged in the water storage tank for washing clothes; and sterilization elements (UV lamps) arranged on one side of the housing to sterilize the water storage tank and the washing tank.

KR200139344 describes a washing machine that comprises UV lighting means on the cover of the washing machine with a dual purpose: to illuminate the interior of the washing machine and additionally sterilize the load by means of UV radiation.

KR20050119338 describes a washing machine with dust collection and clothing sterilization function. The washing machine is characterized by including a filter installed in the drain pipe or exhaust pipe to filter dust, and an ultraviolet lamp to emit sterilizing UV rays into the wash tub.

US 2009/0064422 describes a front-loading sterilizable washing machine that uses ultraviolet radiation and a sterilizable washing method therein. The washing method comprises: supplying water, washing, draining, rinsing and dehydrating and subsequently sterilizing with a UV light source, preferably UVC radiation.

KR101324485 describes a front-loading drum washing machine that includes a tub and a washing drum. A UV light-emitting diode (LED) module is installed in the tub. The UV LED module allows the interior of the tub to be irradiated and have a sterilization effect.

DE7107935 describes an irradiation device for disinfecting clothes in front-loading washing machines and dryers, characterized by the fact that a quartz lamp or a UV ray source is arranged in the door of the washing machine, in such a way that it illuminates the drum during or after the washing or drying process so that the clothing is exposed to intense ultraviolet radiation.

U.S. Pat. No. 5,664,340 describes a front-loading dryer with ultraviolet light designed to kill bacteria and germs that cause mold on clothing. The UV lights are contained within a plate mounted on the dryer door and covered with transparent glass or polycarbonate plastic. When the dryer is turned on, the UV lights will illuminate, beginning the process of removing germs from clothing.

KR20060114101 describes a front-loading drum washer and dryer with sterilization and deodorization function. Sterilization of clothes is carried out at the end, during the drying stage, and a drum wash is carried out. The washing machine door is equipped with ultraviolet ray generating means that radiates ultraviolet rays into the drum. Additionally, the equipment irradiates ozone when drying clothes to completely sterilize and disinfect the clothes in the drum and drum, and eliminate the odor generated by residual contaminants.

Despite these prior efforts, there is still a need for effective alternatives to existing methods for washing and disinfecting textiles that allow their use in different embodiments and provide additional benefits such as energy savings, shorter process times and less damage to textiles by avoiding using high temperatures for disinfection.

### BRIEF DESCRIPTION OF THE INVENTION

The present invention relates to a method of washing textiles that offers the benefit of disinfection of clothing, the wash liquid that is sent to the drain, as well as the components of the washing machine that are in contact with the wash liquid and the load of clothes through the use of ultraviolet light with wavelength C (UVC) and without the need to use high temperatures in the water that is fed to the inlet of the washing machine or inside it for disinfection, by means of an automatic washing machine, for example, top-loading, containing a tub; a basket and infuser located within the tub that can be moved by a motor drive system, power transmission and pulley transmission or a direct drive drive system; wash liquid recirculation system and a source to emit UV light, preferably UVC light, for disinfection. The device that emits UVC light can be, for example, a lamp or LED.

The ultraviolet light generated by the radiation source is within the wavelength range of 100 to 280 nm (short wave or UVC) to eliminate up to 99.9% of viruses and bacteria, reducing the number of microorganisms present on the textiles and preventing their growth inside the household appliance, thus preventing them from being transferred to the textiles to be placed inside in the next cycle.

### BRIEF DESCRIPTION OF THE FIGURES

The illustrative embodiment can be described with reference to the accompanying figures, whereby:

FIG. 1A illustrates the location of the radiation source placed at the bottom of the washing machine tub

FIG. 1B illustrates the lateral view of the location of the radiation source placed at the bottom of the washing machine tub.

FIG. 1C illustrates the top view of the location of the radiation source placed at the bottom of the washing machine tub.

FIG. 2A illustrates an exploded view of the washing machine in the radiation source mode placed on the side of the tub.

FIG. 2B illustrates the top view of the location of the radiation source in the washing machine in the radiation source mode placed on the side of the tub.

FIG. 2C illustrates the lateral view of the location of the radiation source in the washing machine in the radiation source mode placed on the side of the tub.

FIG. 3A illustrates an exploded view of the washing machine in the radiation source mode placed on the infuser.

FIG. 3B illustrates the lateral view of the radiation source in the washing machine in the radiation source mode placed on the infuser.

FIG. 3C illustrates the top view of the location of the radiation source in the washing machine in the radiation source mode placed on the infuser.

FIG. 4A illustrates the top view of the location of the radiation source in the washing machine in the radiation source mode placed at any point below the cover.

FIG. 4B illustrates the lateral view of the location of the radiation source in the washing machine in the radiation source mode placed at any point below the cover.

FIG. 5 illustrates an exploded view of the location of the radiation source in the washing machine in the radiation source mode placed in the recirculation system.

FIG. 6 illustrates an exploded view of the recirculation tube.

### DETAILED DESCRIPTION OF THE INVENTION

The following detailed description is exemplary only and is not intended to limit the described embodiments or the application and uses of the described embodiments. As used herein, the word “exemplary” or “illustrative” means “serving as an example, instance, or illustration”. Any implementation described herein as “exemplary” or “illustrative” should not necessarily be construed as preferred or advantageous over other implementations. All of the implementations described below are exemplary implementations provided to enable those skilled in the art to make or use the embodiments of the disclosure and are not intended to limit the scope of the disclosure. In addition, for purposes of description herein, the terms “end”, “upper”, “lower”, “right”, “left”, “vertical”, “horizontal”, “lateral”, “longitudinal” and derivatives thereof are related to the invention as shown in the figures. In addition, there is no intention to be subject to any explicit or implicit theory presented in the technical field above, background, brief summary or the following detailed description. It should also be understood that the specific devices and processes illustrated in the accompanying drawings, and described in the following description, are merely exemplary embodiments of the inventive concepts defined in the attached claims.

The illustrations generally show non-limiting aspects of the systems and methods of the present description. Although the various aspects of the present descriptions of the devices should not be construed in any way as limiting the description. In addition, any modifications, concepts, and applications of aspects of the description are to be construed by those skilled in the art as comprised by, but not limited to, the illustrations and descriptions herein. Several modifications, equivalents, variants and alternatives, however, will be readily apparent to those skilled in the art. Any and all such modifications, variants, equivalents and alternatives are intended to fall within the spirit and scope of the present description.

As used herein, the singular articles “a”, “an”, and “the” include plural referents unless expressly and unambiguously limited to one referent. However, it is to be understood that the description may assume several alternatives, variations, and sequences of steps, except where expressly stated otherwise. It is also to be understood that the specific devices and processes illustrated in the accompanying drawings and described in the following description are non-limiting representations of several aspects of the description. Therefore, the specific dimensions and other physical characteristics related to the aspects described herein are not intended to be and should not be considered as limiting.

Unless otherwise indicated, all ranges or ratios described herein are meant to encompass any and all sub-ranges or sub-ratios incorporated herein. For example, an established range or ratio of “1 to 10” should be considered to include any and all sub-ranges between (and including) the minimum value of 1 and the maximum value of 10; that is, all sub-ranges or sub-ratios that start with a minimum value of 10 or less, such as, but not limited to, 1 to 6.1, 3.5 to 7.8, and 5.5 to 10. Unless otherwise indicated, all numbers expressing dimensions, quantities of ingredients, flow rates, pressures, etc., used in the description and claims, are to be construed as modified in all instances by the term “approximately”. For the purposes of the present description, the term “approximately” determines a range of ±10%, whereby if a value of “approximately 30%” is specified, the value is within a range of 27% to 33%.

The present invention relates to a household appliance and a method of disinfecting textiles with UVC technology using said household appliance. Said household appliance may include, for example, a washing machine comprising a cabinet on which a tub, a basket (not illustrated) and an agitator are arranged, which may be a conventional agitator such as a propeller or an agitator using cones, wheels, fins, low discs or similar elements, such as an infuser, located within the tub, which can be moved by any motive means known in the art, for example, a motor drive system, power transmission and pulley transmission or a direct drive impeller system and a cabinet cover; a system for recirculating the washing liquid to the basket is included, which includes a recirculation tube and a recirculation pump and a radiation source for disinfection. The radiation source, in a preferred embodiment, emits UVC light and can be, for example, a lamp or LED that can be located in the recirculation system, at the bottom of the tub, on the side of the tub, on the infuser, or at any point under the cabinet cover.

The sequence of steps of the washing and rinsing method, according to a preferred embodiment of the invention comprises:
1. Arranging the load of laundry by spinning the basket at a predetermined speed in a range of 100 to 500 revolutions per minute. This step may be optional.
2. Starting filling the wash basket to the first wash level in the operating range of 8 to 30 cm of water column; during this filling, the additives, for example, detergent, other stain removers, etc., from the washing phase are dispensed and hot and cold water are combined to obtain the selected temperature level between 15° C. and 60° C.
3. Performing load sensing using a shaking pattern with an are of rotation of approximately 360° at 60 strokes per minute for approximately 0.2 to 5 minutes. In this stage, the resistance that the washing load opposes to the drive system is measured, which is a function of the size and type of load and other parameters and serves to assign the corresponding water level.

### Washing Phase

4. Depending on the size and type of clothing, the predetermined amount of water is assigned, maintaining the combination of proportions of hot and cold water according to the selected temperature level, while the basket is filled with water, the load is gently shaken in a rotating arc between 180° 720° and a speed of 100 to 150 revolutions per minute.
5. Intense agitation starts with an arc of rotation between 180° to 1080° at 20 to 100 revolutions per minute, rotating clockwise and changing counterclockwise.
6. A stirring pattern of 90° to 540° arc of rotation is run at 30 to 100 revolutions per minute to balance the load.
7. The agitation to balance the load ends and the draining and centrifuging of the load begins to extract the wash liquid.

### Rinsing Phase

8. It is filled with fresh water to the same level as it was washed.
9. It is agitated with the same intense agitation pattern used in the washing phase, followed by 1 to 10 minutes with the laundry settling agitation pattern.
10. All the water is drained and the load is centrifuged.

Regarding this method, the use of UVC light has an effect in steps 4, 5 and 6 of the washing phase and/or in steps 8 and 9 of the rinsing phase. This method is applied equally to any type of washing system, for example, agitator, infuser or propeller.

The method described above can have different embodiments, as detailed below.

In a first embodiment, the radiation source is turned on 100% of the time of the washing and rinsing phases.

In a second embodiment, the radiation source is turned on at controlled time intervals between 0 and up to approximately 50 min during the washing phase, while the radiation source remains turned on during the rinsing phase.

In a third embodiment, the radiation source is turned on at controlled time intervals between 0 and up to approximately 50 min during the rinsing phase, while the radiation source remains turned on during the washing phase.

In a fourth embodiment, the radiation source is turned on at controlled time intervals between 0 and up to approximately 50 min during the washing and rinsing phase.

In a fifth embodiment, the radiation source remains turned on during the washing phase, while the radiation source remains off during the rinsing phase.

In a sixth embodiment, the radiation source remains turned on during the rinsing phase, while the radiation source remains off during the washing phase.

In a seventh embodiment, the radiation source is turned on at controlled time intervals between 0 and up to approximately 50 min during the washing phase, while the radiation source remains off during the rinsing phase.

In an eighth embodiment, the radiation source is turned on at controlled time intervals between 0 and up to approximately 50 min during the rinsing phase, while the radiation source remains off during the washing phase.

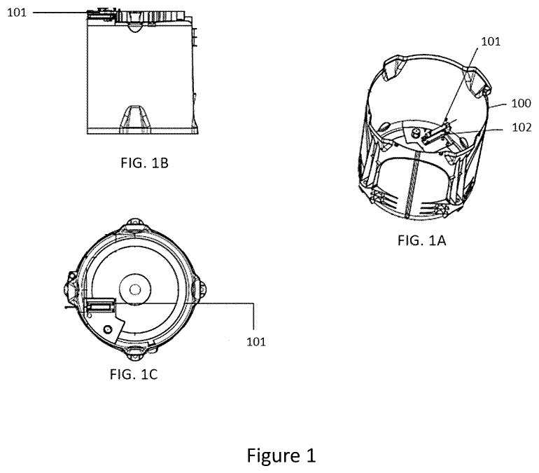

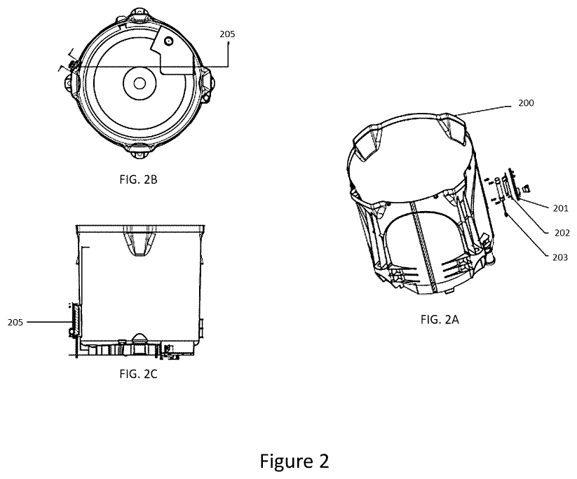

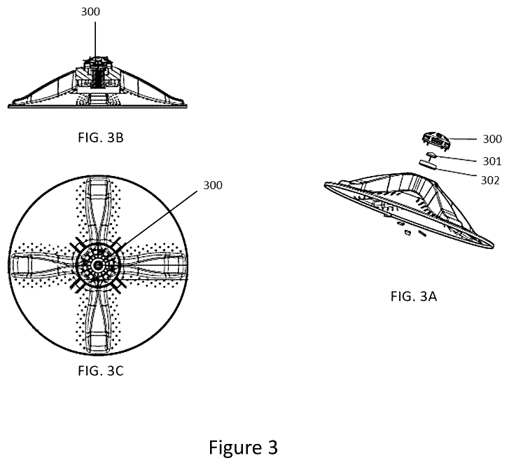

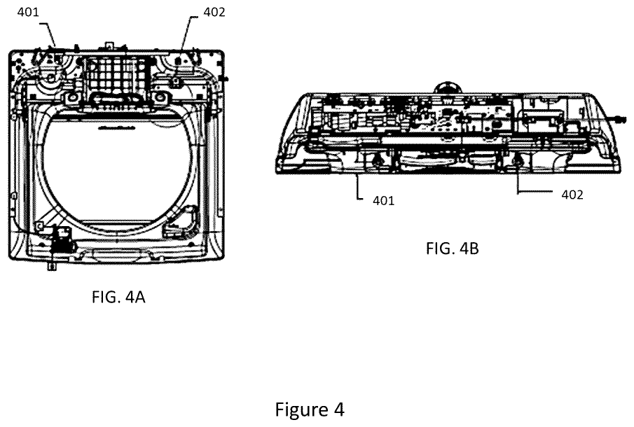

All of the previously described embodiments apply to the location of the radiation source (101) at the bottom of the tub (100) in the washing machine as illustrated in FIG. 1A, FIG. 1B and FIG. 1C where said radiation source (101) is covered by a casing (102), the location of the radiation source (203) on the side of the tub (200) of the washing machine as illustrated in FIG. 2A, FIG. 2B and FIG. 2C where the radiation source (203) is covered by a protective barrier (202) and this in turn is covered by a casing (201), the location of the radiation source (301) on the infuser as illustrated in FIG. 3A, FIG. 3B and FIG. 3C where the radiation source (301) is located on a base (302) and covered by a casing (300), which in a preferred embodiment may comprise a seal to seal and prevent leaks and be made of a heat resistant material, for example, glass, and the location of the radiation source (401, 402) under the cover as illustrated in FIG. 4A and FIG. 4B.

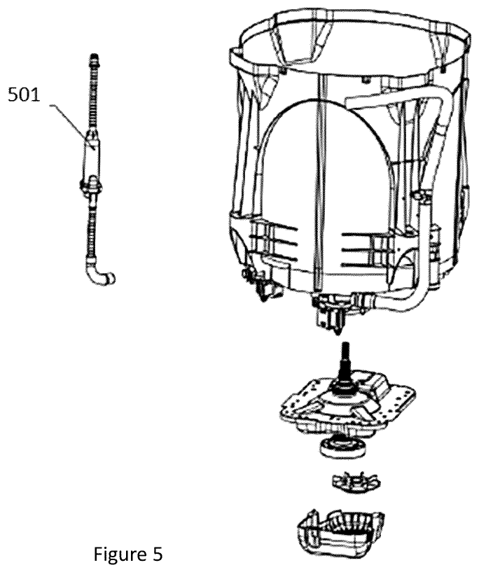

For the location of the radiation source (501) in the recirculation system as illustrated in FIG. 5, the following embodiments are available:
1. The intermittent turning on of the radiation source takes effect from the end of the load-sensing stage of the washing phase until the beginning of the rinsing phase.
2. The intermittent turning on of the radiation source takes effect from the end of the load-sensing stage of the washing phase until the beginning of the rinsing phase, with continuous turning on of the radiation source from the start of filling with fresh water for the rinsing phase until the end of said phase.
3. The intermittent turning on of the radiation source takes effect from the end of the load-sensing stage during the washing phase until the beginning of the rinsing phase, with intermittent turning on of the radiation source from the start of filling with fresh water for the rinsing phase until the end of said phase.
4. The continuous turning on of the radiation source takes effect from the end of the load-sensing stage during the washing phase until the beginning of the rinsing phase, without turning on the radiation source during the rinsing phase.
5. The continuous turning on of the radiation source takes effect from the end of the load-sensing stage during the washing phase until the beginning of the rinsing phase, with continuous turning on of the radiation source from the start of filling with fresh water for the rinsing phase until the end of said phase.
6. The continuous turning on of the radiation source takes effect from the end of the load-sensing stage during the washing phase until the beginning of the rinsing phase, with intermittent turning on of the radiation source from the start of filling with fresh water for the rinsing phase until the end of said phase.

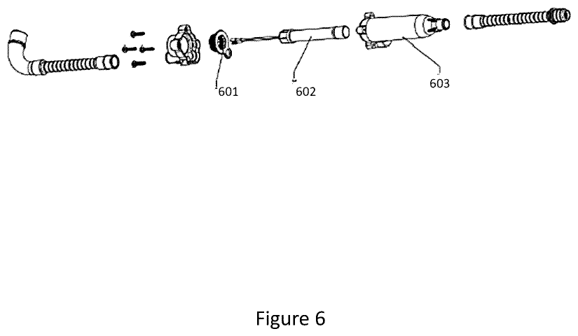

In these embodiments, the activation of the radiation source is conditioned on the use of the recirculation of the washing liquid and where the turning on of the radiation source is carried out in parallel with the turning on of the recirculation pump Recirculation, in a preferred embodiment, is carried out by activating the recirculation pump to move the fluid contained in the basket of the household appliance, from the bottom of the basket to the top of it, forcing it to pass around said source, illustrated in FIG. 6 where the radiation source (602) is located inside a casing (603) embedded within the recirculation tube and optionally closed by a gasket (601), ensuring that the microorganisms in the fluid are eliminated by light radiation UVC applied before returning to the basket.

The method of the present invention makes it possible to efficiently reduce the number of microorganisms on textiles. In addition, the proposed method provides a flexible alternative to existing methods, since it allows its application in different embodiments, at the user's convenience.

A person skilled in the art can modify the structure described herein. However, it should be noted that this description relates to preferred embodiments of the invention, and is provided for illustrative purposes only, and should not be understood as limiting the invention. All obvious modifications in the spirit of the invention, such as changes in the shape, material, and dimensions of the elements that make up the invention, should be considered within the scope of the attached claims.

The invention has been described in an illustrative manner, and it should be understood that the terminology used herein is intended to correspond to the nature of the words of the description rather than to provide a limitation.

Obviously, many modifications and variations of the present invention are possible in light of the above teachings. Therefore, it should be understood that within the scope of the invention described, the invention may be practiced otherwise than as specifically described.

1. A system for washing and disinfecting textiles in a household appliance that comprises:
a tub with a basket inside the tub, the basket being capable of receiving the textiles;
a driving means to drive the basket;
a recirculation circuit that comprises a recirculation tube and a recirculation pump to recirculate a liquid coming from a bottom of the tub and directed towards an interior of the basket; and
a source of UVC radiation arranged inside the recirculation tube to disinfect the washing liquid.

2. The system for washing and disinfecting textiles in a household appliance according to claim 1, wherein the radiation source is selected from a lamp or LED, wherein the radiation source is located within a casing embedded within the recirculation tube and wherein the appliance comprises a cabinet cover.

3. The system for washing and disinfecting textiles in a household appliance according to claim 2, wherein the radiation source is located at the bottom of the tub, on a side of the tub, on an agitator of the household appliance, or at any point below the cabinet cover, to disinfect textiles and components of the household appliance in contact with the washing liquid.

4. The system for washing and disinfecting textiles in a household appliance according to claim 2, wherein the radiation source housing comprises a packaging.

5. The system for washing and disinfecting textiles in a household appliance according to claim 2, wherein the radiation source has a wavelength within the range of approximately 100 to 280 nm.

6. The system for washing and disinfecting textiles in a household appliance according to claim 1, wherein the appliance is a washing machine and wherein an agitator of the appliance is an infuser or a propeller.

7. A method of washing and disinfecting textiles in a household appliance having a tub with a basket inside the tub and a UVC radiation source arranged within a recirculation tube of a recirculation circuit, comprising:
turning on the UVC radiation source;
circulating a washing liquid coming from a bottom of the basket towards an interior of the basket through the recirculation circuit to irradiate the liquid through the UVC radiation source; and
turning off the radiation source.

8. A method of washing and disinfecting textiles in a household appliance that has a wash basket, an agitator, a cabinet cover, and a UVC radiation source installed at a bottom of the tub, on a side of the tub, above the agitator, or at any point below the cover, comprising:
filling the wash basket with water;
placing the textiles inside the basket;
turning on the radiation source to irradiate the textiles;
starting washing and rinsing phases of the textiles;
turning off the radiation source; and
draining the liquid from washing the textiles;
wherein the radiation source also irradiates components of the household appliance that are in contact with the washing liquid.

9. The method of washing and disinfecting textiles in a household appliance for washing and disinfecting textiles according to claim 7, wherein the radiation is preferably UVC radiation.

10. The method of washing and disinfecting textiles in a household appliance for washing and disinfecting textiles according to claim 8, wherein the radiation source is turned on continuously during the washing phase until a beginning of the rinsing phase, and is turned off during the rinsing phase.

11. The method of washing and disinfecting textiles in a household appliance for washing and disinfecting textiles according to claim 8, wherein the radiation source is turned on continuously during the washing phase until a beginning of the rinsing phase, and is turned on intermittently during the rinsing phase until an end of said phase.

12. The method of washing and disinfecting textiles in a household appliance for washing and disinfecting textiles according to claim 8, wherein the radiation source is turned on continuously during the washing and rinsing phases.

13. The method of washing and disinfecting textiles in a household appliance for washing and disinfecting textiles according to claim 8, wherein the radiation source is turned on continuously during the rinsing phase until an end thereof, and is turned on intermittently during the washing phase until a start of the rinsing phase.

14. The method of washing and disinfecting textiles in a household appliance for washing and disinfecting textiles according to claim 8, wherein the radiation source is turned on intermittently during the washing and rinsing phases.

15. The method of washing and disinfecting textiles in a household appliance for washing and disinfecting textiles according to claim 8, wherein the radiation source is turned on intermittently during the washing phase until a beginning of the rinsing phase, and is turned off during the rinsing phase.

16. The method of washing and disinfecting textiles in a household appliance for washing and disinfecting textiles according to claim 11, wherein the intermittent turning on of the radiation source is carried out at intervals from one minute to approximately fifty minutes and turns off from one minute to approximately fifty minutes.

17. The method for washing and disinfecting textiles in a household appliance according to claim 7, wherein the turning on of the radiation source is carried out in parallel with turning on a recirculation pump of the recirculation circuit.

18. The method of washing and disinfecting textiles in a household appliance for washing and disinfecting textiles according to claim 8, wherein the radiation source is turned on intermittently during at least one of: the washing phase and the rinsing phase.

19. The method of washing and disinfecting textiles in a household appliance for washing and disinfecting textiles according to claim 18, wherein the intermittent turning on of the radiation source is carried out at intervals from one minute to approximately fifty minutes and turns off from one minute to approximately fifty minutes.
